(19) United States
(12) Patent Application Publication
(10) Pub. No.: US 2012/0003198 A1
(43) Pub. Date: Jan. 5, 2012

(54) Lutein/Zeaxanthin for glare protection

(75) Inventors: Felix Barker, Wyncote, PA (US); Regina Goralczyk, Grenzach-Wyhlen (DE); Wolfgang Schalch, Bottmingen (CH)

(73) Assignee: DSM IP ASSETS B.V., TE Heerlen (NL)

(21) Appl. No.: 13/137,729

(22) Filed: Sep. 8, 2011

Related U.S. Application Data
(63) Continuation of application No. 10/503,101, filed on Jul. 28, 2004, now abandoned.

(30) Foreign Application Priority Data
Jan. 30, 2002 (EP) 02.001909.7

(51) Int. Cl. A61K 36/06; C07C 69/753; A61K 31/047; A61K 31/215; A61K 31/23; A61K 31/231; A61K 31/232; A61K 31/355; A61K 31/375; A61K 31/195; A61K 36/45; A61K 31/315; A61P 27/02; A61P 39/06; C07C 35/21

(52) U.S. Cl. 424/93.51; 568/816; 560/259; 560/194; 554/229; 554/223; 554/224; 514/729; 514/546; 514/548; 514/552; 514/549; 514/458; 514/474; 514/561; 424/732; 514/494

(57) ABSTRACT

The invention relates to the improvement of visual performance, particularly of visual performance in the darkness, by administration of a colorant that is capable of being incorporated into eye tissue and/or causing yellowing of eye tissue, especially carotenoids, such as lutein and zeaxanthin.

This application is a continuation of application Ser. No. 10/503,101 filed Jul. 28, 2004, which in turn is the national phase of International Application Ser. No. PCT/EP03/00656 which claims priority of EP 02001909.7 filed Jan. 30, 2002, the entire content of each of which is hereby incorporated by reference in this application.

The present invention relates to the improvement of visual performance. More particularly, the present invention relates to the improvement of visual performance in the darkness, e.g., when steering a vehicle or aircraft under low or dim light conditions such as at night or dawn, or when steering a vehicle in tunnels.

In accordance with the present invention it has been found that administration of a colorant that is capable of being incorporated into eye tissue and/or causing yellowing of eye tissue results in an improvement of visual performance. A common feature of such colorants is that they when deposited in the eye tissue, particularly the retina, provide a yellow filter that absorbs blue light. Blue light is supposed to be potentially damaging to the retina. Examples of colorants that can be used in accordance with the present invention are carotenoids, such as lutein, zeaxanthin, mesozeaxanthin, astaxanthin, or esters thereof, or canthaxanthin, or mixtures of the foregoing, as well as compounds having vitamin A activity, or precursors thereof. Administration of lutein and zeaxanthin has been found to lower the risk for developing age-related macular disease (AMD). However, these compounds are useful to improve visual performance also in the absence of AMD. Accordingly, in one aspect the present invention relates to the use of colorant that is capable of being incorporated into eye tissue and/or causing yellowing of eye tissue, particularly a carotenoid such as a compound selected from lutein, zeaxanthin, mesozeaxanthin, astaxanthin, or esters thereof, or canthaxanthin, or mixtures of the foregoing, and/or a compound having vitamin A activity, or precursor thereof, in the manufacture of a composition for improving visual performance in the darkness. In another aspect, the present invention relates to a method of improving visual performance in the darkness, by administration of an effective amount of one or more of the aforesaid colorants.

In a further embodiment, the present invention relates to the use of a the aforementioned colorants in combination with an anti-oxidant selected from vitamin E, vitamin C, a zinc or anorganic selenium salt such as selenophosphates or sodium selenite or sodium selenateor seleno aminoacids such as L-selenomethionine, or selenized yeast such as brewer's yeast or baker's yeast (Saccharomyces cerevisiae) containing or enriched in selenium, and bilberry extract containing approx 20 to 30 anthocyanosides, or mixtures thereof in the manufacture of a composition for improving visual performance.

Esters of lutein, zeaxanthin, mesozeaxanthin or astaxanthin are preferably esters of saturated alkanoic acids such as acetic, propionic, palmitic, stearic and succinic acid, mono-unsaturated fatty acids such as oleic acid, and poly-unsaturated fatty acids such as linolic, docosahexaenoic and arachidonic acid.

Examples of compounds having vitamin A-activity or precursors thereof are retinol and esters thereof, such as retinyl palmitate; α- and β-carotene, β-cryptoxanthin, and lycopene. Examples of zinc salts are zinc salts of mineral acids such as zinc sulfate, or of organic acids such as zinc orotate. Examples of organic selenium salts are selenophosphates or sodium selenite or sodium selenate.

The term “vitamin E” refers to natural or racemic α-tocopherol as well as esters thereof such as the acetate. The term “vitamin C” comprises ascorbic acid and esters and salts thereof such as ascorbyl palmitate and sodium ascorbyl phosphate.

The term “visual performance” as used herein refers to visual functions such as acuity, contrast sensitivity, dark adaptation, glare recovery, photostress recovery, retinal sensitivity, blue cone sensitivity, color vision and visual field. Of particular interest is recovery from and the reduction of the physiological effects of glare, especially glare caused by blue light, e.g., when driving in the darkness, i.e. at night or dawn, or in tunnels. Another visual function that may be improved in accordance with the present invention is accuracy in target shooting.

The term “eye tissue” comprises retina, lens, vitreous, retinal pigment epithelium, iris and ciliary body. The term “composition as used herein denotes any composition that is suitable for administration to the human body, such as pharmaceutical preparations, food or beverage.

A pharmaceutical preparation in accordance with the present invention for improvement of visual performance may be in any form that is conventional for oral administration, e.g. in solid form such as tablets including effervescent tablets, or soft or hard shell capsules, or in liquid form, such as solutions or suspensions, preferably oily suspension. Besides the active ingredients the pharmaceutical preparation may contain conventional pharmaceutical carrier material, additives and adjuvants, which include water, gelatin, vegetable gums, sugars, vegetable oils, polyalkylene glycols, flavoring agents, preservatives, stabilizers, emulsifying agents, buffers and the like. The medicaments may be in the form of controlled (delayed) release formulations. For the purpose of the invention the colorants as well as optional ingredients as defined earlier hereinabove may be incorporated in food or beverages, such as bakery items, e.g., cake and cookies, lemonades and fruit juices.

In a preferred aspect, the invention relates to the use of colorants as defined earlier hereinabove in the manufacture of a medicament, a food or beverage for reducing glare or promoting recovery from glare in driving at night. Preferably, a combination of lutein and zeaxanthin is used. In such combination these compounds are preferably used in a ratio of 0.1-1.0:1.0-0.1 parts by weight.

In solid pharmaceutical preparations, the compounds selected from lutein, zeaxanthin, mesozeaxanthin, astaxanthin, β-cryptoxanthin or esters thereof, or canthaxanthin are suitably present in an amount from about 0.1 mg to about 500 mg, preferably from about 1 mg to about 100 mg per dosage unit. In liquid formulations, the aforesaid ingredients are suitably present in an amount of from about 0.1 to about 5 percent by weight based upon the total weight of the composition. If vitamin E is present, its amount is suitably from about 10 to about 1000 mg per dosage unit in solid formulations and from about 0.1 to about 500 mg in liquid formulations. In liquid formulations vitamin E may serve as a carrier for other lipophilic components of the formulations in accordance with the invention and may comprise 99.9-50% percent by weight based upon the total weight of the composition. If vitamin C is present, its amount is suitably from about 10 to about 1000 mg per dosage unit. Compounds having vitamin A activity or precursors thereof may be present in amounts providing a vitamin A activity of from about 100 to about 10000 International Units per dosage unit. Zinc may be present in an amount of 1 to 100 mg (based on elementary zinc) per dosage unit. Selenium may be present in an amount of 10 to 200 microgram mg (based on elementary selenium) per dosage unit. Bilberry extract may be used in amounts of 50 to 150 mg (usually containing 20 to 30% anthocyanosides) per dosage unit.

Preferred solid pharmaceutical preparations comprise, per dosage unit, about 6 mg to about 12 mg of lutein, zeaxanthin, mesozeaxanthin, astaxanthin β-cryptoxanthin or esters thereof, or canthaxanthin, or mixtures of the foregoing; about 200 mg of vitamin E; and 1 mg to about 10 mg of zinc, and, optionally, about 1000 International Units of vitamin A and further optionally, about 1 mg to about 10 mg of β-carotene. Thus, in a further aspect, the present invention also relates to such preferred solid pharmaceutical preparations.

A suitable daily dosage of the ingredients, lutein, zeaxanthin, mesozeaxanthin, astaxanthin β-cryptoxanthin or esters thereof, or canthaxanthin in a pharmaceutical preparation prepared in accordance with the present invention or contained in any food or beverage is, e.g., within the range of from 0.001 mg per kg body weight to about 20 mg per kg body weight. More preferred is a daily dosage of about 0.01 to about 10 mg per kg body weight, and especially preferred is about 0.1 to 1.0 mg per kg body weight per day, based upon the total weight of these components in their unesterified form.

The invention is illustrated further by the Examples given below:

### EXAMPLE 1

A soft gelatin capsule may be prepared comprising the following ingredients:

| Ingredient | Amount per Capsule |
|---|---|
| Lutein | 10 mg |
| Lecithin | 50 mg |
| Soy bean oil | 200 mg |

### EXAMPLE 2

A soft gelatin capsule may be prepared comprising the following ingredients:

| Ingredient | Amount per Capsule |
|---|---|
| Lutein | 10 mg |
| Zeaxanthin | 10 mg |
| Lecithin | 50 mg |
| Soy bean oil | 200 mg |

### EXAMPLE 3

A soft gelatin capsule may be prepared comprising the following ingredients:

| Ingredient | Amount per Capsule |
|---|---|
| Lutein | 6 mg |
| Zeaxanthin | 6 mg |
| Vitamin E (α-d,l-tocopherol) | 200 mg |
| Vitamin C | 500 mg |
| Lecithin | 50 mg |
| Soy bean oil | 200 mg |

### EXAMPLE 4

A soft gelatin capsule may be prepared comprising the following ingredients:

| Ingredient | Amount per Capsule |
|---|---|
| Lutein | 12 mg |
| Vitamin E (α-d,l-tocopherol) | 200 mg |
| Vitamin C | 500 mg |
| Lecithin | 50 mg |
| Soy bean oil | 200 mg |

### EXAMPLE 5

A soft gelatin capsule may be prepared comprising the following ingredients:

| Ingredient | Amount per Capsule |
|---|---|
| Zeaxanthin | 12 mg |
| Vitamin E (α-d,l-tocopherol) | 200 mg |
| Vitamin C | 500 mg |
| Lecithin | 50 mg |
| Soy bean oil | 200 mg |

### EXAMPLE 6

A soft gelatin capsule may be prepared comprising the following ingredients:

| Ingredient | Amount per Capsule |
|---|---|
| Lutein | 6 mg |
| Zeaxanthin | 6 mg |
| β-Carotene | 6 mg |
| Vitamin E (α-d,l-tocopherol) | 200 mg |
| Vitamin C | 500 mg |
| Zinc (as orotate) | 7.5 mg |
| Lecithin | 50 mg |
| Soy bean oil | 200 mg |

### EXAMPLE 7

A soft gelatin capsule may be prepared comprising the following ingredients:

| | Amount per | |
|---|---|---|
| Ingredient | Capsule | |
| Lutein | 6 | mg |
| Zeaxanthin | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

### EXAMPLE 8

A soft gelatin capsule may be prepared comprising the following ingredients:

| | Amount per | |
|---|---|---|
| Ingredient | Capsule | |
| Lutein | 6 | mg |
| Zeaxanthin | 6 | mg |
| β-Carotene | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

1. The use of a colorant that is capable of being incorporated into eye tissue and/or causing yellowing of eye tissue, in the manufacture of a composition for the improvement of visual performance in the darkness.

2.-27. (canceled)

28. A method of promoting recovery from the physiological effects of glare which comprises administering to a healthy person in need of such treatment an effective amount of a carotenoid in a daily dosage within the range of from about 0.1 mg per kg body weight to 1.0 mg per kg body weight, based on the total weight of the carotenoid devoid of any ester group,
wherein the carotenoid is selected from the group consisting of: lutein, zeaxanthin, mesozeaxanthin, astaxanthin or esters thereof, or mixtures thereof; and
wherein the esters thereof are selected from the group consisting of esters of acetic acid, propionic acid, palmitic acid, stearic acid, succinic acid, oleic acid, linoleic acid, docosahexaenoic acid, and arachidonic acid.

29. The method according to claim 28 wherein the carotenoid is lutein or zeaxanthin, or ester of lutein or zeaxanthin, or any combination thereof.

30. The method according to claim 29, wherein the carotenoid is a combination of lutein and zeaxanthin.

31. The method according to claim 28 which comprises additionally administering an anti-oxidant selected from vitamin E, vitamin C, a zinc or selenium salt, or a selenium aminoacid, selenized yeast and bilberry extract, or mixtures thereof.

32. The method according to claim 28 wherein the carotenoids are combination of lutein and zeaxanthin in a ratio of 0.1-1.0:1.0-0.1 parts by weight.

33. The method according to claim 28, wherein recovery from glare is promoted for a situation selected from the group consisting of: steering a vehicle at night or dawn; steering an aircraft at night or dawn; and steering a vehicle in a tunnel.

34. The method according to claim 28 wherein the daily dosage of carotenoid is selected from the group consisting of: 10-12 mg; 10 mg; 12 mg; and 20 mg.

35. A method of reducing the physiological effects of glare which comprises administering to a healthy person in need of such treatment an effective amount of a carotenoid in a daily dosage within the range of from about 0.1 mg per kg body weight to 1.0 mg per kg body weight, based on the total weight of the carotenoid devoid of any ester group,
wherein the carotenoid is selected from the group consisting of: lutein, zeaxanthin, mesozeaxanthin, astaxanthin or esters thereof, or mixtures thereof; and
wherein the esters thereof are selected from the group consisting of esters of acetic acid, propionic acid, palmitic acid, stearic acid, succinic acid, oleic acid, linoleic acid, docosahexaenoic acid, and arachidonic acid.

36. The method according to claim 35 wherein the carotenoid is lutein or zeaxanthin, or ester of lutein or zeaxanthin, or any combination thereof.

37. The method according to claim 36, wherein the carotenoid is a combination of lutein and zeaxanthin.

38. The method according to claim 35 which comprises additionally administering an anti-oxidant selected from vitamin E, vitamin C, a zinc or selenium salt, or a selenium aminoacid, selenized yeast and bilberry extract, or mixtures thereof.

39. The method according to claim 35 wherein the carotenoids are combination of lutein and zeaxanthin in a ratio of 0.1-1.0:1.0-0.1 parts by weight.

40. The method according to claim 35, wherein recovery from glare is promoted for a situation selected from the group consisting of: steering a vehicle at night or dawn; steering an aircraft at night or dawn; and steering a vehicle in a tunnel.

41. The method according to claim 35 wherein the daily dosage of carotenoid is selected from the group consisting of: 10 mg, 12 mg, and 20 mg.
